(19) United States
(12) Patent Application Publication
(10) Pub. No.: US 2012/0270747 A1
(43) Pub. Date: Oct. 25, 2012

(54) METHOD OF PREDICTING RISK OF PRE-TERM BIRTH

(75) Inventor: Michal A. Elovitz, Wynnewood, PA (US)

(73) Assignee: The Trustees of the University of Pennsylvania, Philadelphia, PA (US)

(21) Appl. No.: 13/504,781

(22) PCT Filed: Oct. 28, 2010
(86) PCT No.: PCT/US2010/054377

§ 371 Date: Jul. 11, 2012

Related U.S. Application Data
(60) Provisional application No. 61/256,066, filed on Oct. 29, 2009.

(51) Int. Cl. G01N 33/566; G01N 21/76; G01N 21/64; C12Q 1/68; C40B 30/04

(52) U.S. Cl. 506/9; 436/501; 435/7.92; 435/6.12; 435/6.11

(57) ABSTRACT

A method for diagnosing, or differentially diagnosing, an increased risk of pre-term birth (PTB) involves detecting or measuring increased expression of a biomarker Soluble E-cadherin (SE-CAD) in a biological sample from a mammalian subject, particularly in the urine, cervicovaginal fluid or blood. An increased level of expression of SE-CAD above the level of expression in the same sample of a healthy mammalian subject is an indication of a diagnosis of increased risk of PTB. Such diagnosis may further involve identify other clinical symptoms of PTB or PTL. Additionally the method may use additional biomarkers, such as fetal fibronectin.

### BACKGROUND OF THE INVENTION

Pre-term birth (PTB) is the leading cause of neonatal mortality and a significant contributor to neonatal morbidity. In the United States, approximately 12% of all live births are born preterm, i.e., before 37 weeks of gestational age. The incidence of PTB has not declined and, in fact, has demonstrated an upward trend. The extreme cost of PTB resides not only in the immediate neonatal care but also in long-term care of lasting morbidities resulting from prematurity) The care of preterm infants consumes a significant proportion of health care costs for children. Recent data suggest that there are more long-term sequelae from PTB than previously recognized including significant neurobehavioral abnormalities as these children reach school age. Effective prevention or treatment of PTB could significantly lower neonatal mortality and morbidity as well as health care costs. Therapies that target pregnant women at greatest risk for PTB are urgently needed. However, these types of interventions and therapeutic strategies cannot begin until we are able to reliably identify which women and infants are truly at greatest risk. To date, attempts at predicting which women will have PTB have not been successful. While some biomarkers have demonstrated high specificity, they lack sensitivity and positive predictive value making them a poor tool for risk stratification. It is of great clinical interest to identify biomarkers (either proteins in body fluids or in genetic ‘tendencies’) for the prediction of PTB allowing risk stratification, treatment and care to the highest risk group, while an identified ‘lower risk’ group can be spared unnecessary treatment, hospital stays, and concern of having a preterm neonate. Identifying those women at greatest risk for PTB will undoubtedly lead to new preventative strategies.

The identification of a short cervix has proven to be the strongest predictor of PTB^{6, 7} but still lacks significant test characteristics that would make it a useful screening test for the general population. Even when measured serially, short cervix has a low sensitivity for predicting PTB.⁹ In addition, the prevalence of a very short cervix (<15 mm) is quite low, limiting the use of this measure as a screening test for PTB. Similarly, a positive fetal fibronectin (FFN) is strongly associated with PTB, while a negative test is a strong predictor of the pregnancy continuing for at least 14 more days.⁶ The poor positive predictive value of FFN, as well as variability across populations, limits its use to the general population, suggesting that FFN and CL may just represent the same biological pathway; these two biomarkers are not additive in their prediction of PTBs.⁶ The presence of bacterial vaginosis (BV) has been associated with PTB.¹⁰ But, in regards to BV, the data is conflicting. The presence of BV appears to be associated with an increased risk in some but not all populations;^{11, 12} yet, treatment of BV in asymptomatic women conferred no change in the rate of PTB.¹³ Screening women for BV to prevent PTB is currently only recommended for high risk women.

Although some progress has been made to date, there is still no reliable and definitive marker for pre-term birth. There remains a need in the art for more accurate and sensitive diagnostic or predictive assays for PTB.

### SUMMARY OF THE INVENTION

In one aspect, a method for diagnosing, or predicting the likelihood of occurrence of, or increased risk of, pre-term birth (PTB) is provided comprising measuring the level of expression of a biomarker, soluble E-cadherin (SE-CAD), in a biological sample from a pregnant mammalian subject, wherein an increased level of expression of SE-CAD above the level of expression in a predetermined control is an indication of a diagnosis or likelihood of increased risk of pre-term birth.

In another aspect, a method for screening a population of pregnant women for premature cervical remodeling is provided comprising measuring the level of expression of a biomarker, soluble E-cadherin (SE-CAD), in a biological sample from a pregnant mammalian subject, wherein an increased level of expression of SE-CAD above the level of expression in a predetermined control indicates cervical changes related to an increased risk of PTB.

In another aspect is provided use of a diagnostic reagent to detect or measure SE-CAD in a biological sample for diagnosis of, or predicting the likelihood of occurrence of, or increased risk of, pre-term birth (PTB).

Other aspects and advantages of the invention are described further in the following detailed description of the preferred embodiments thereof.

### BRIEF DESCRIPTION OF THE DRAWINGS

FIG. 1 is a bar graph showing the level of SE-CAD in maternal serum of mice. Levels were measured at 2, 4 and 6 hours after intrauterine infusion of LPS or saline using a mouse model of inflammation-induced pre-term birth.

FIG. 2 is a scatter plot showing SE-CAD levels in maternal serum of women presenting in pre-term labor before 30 weeks gestational age. The squares represent data points of women who delivered at <34 weeks. The triangles represent data points of women who delivered at >34 weeks.

FIG. 3 is a bar graph showing the SE-CAD levels in serum of a control group (who delivered at term), women with preeclampsia who did not have preterm labor or delivery, and women with pre-term labor but who did not have pre-term birth.

FIG. 4 is a scatter plot showing the expression of several immune biomarkers in maternal serum of women with pre-term labor who did have pre-term birth (PT) and who did not have pre-term birth (T).

### DETAILED DESCRIPTION OF THE INVENTION

The present invention answers the need in the art by providing novel methods for diagnosing or predicting the likelihood of occurrence, or increased risk of, pre-term birth utilizing as a novel biomarker of cervical remodeling, soluble E-cadherin (SE-CAD), in both symptomatic and asymptomatic women.

### A. Definitions

“Increased likelihood” of pre-term birth, as used herein, means an increase in the risk or probability that the subject will develop pre-term birth as compared to a predetermined control. In one embodiment, increased likelihood means a 5-6 fold increase over the control level.

“Pre-term birth” (PTB) as used herein means the birth of a baby at less than 37 weeks gestational age. In one embodiment, late pre-term birth means birth of a baby between 34-37 weeks gestational age. In another embodiment, early pre-term birth means birth of a baby at less than 34 weeks gestational age.

“Pre-term labor” (PTL) as used herein, means the onset of labor symptoms at less than 37 weeks gestational age. Labor symptoms include cramps or contractions, watery discharge from the vagina, backache, severe pelvic pressure, and blood from the vagina. Pre-term labor may or may not progress into pre-term birth. In one embodiment, PTL means labor that begins on or after 22 weeks gestational age.

“Diagnosis” as used herein, means determining, screening, or identifying the presence or level of expression of a biomarker in a biological sample that indicates that a subject has an increased likelihood of developing a disease.

“Diagnosis of PTB as used herein means determining, screening, or identifying the presence or level of expression of soluble E-cadherin in a biological sample that indicates that a subject has an increased likelihood of developing PTB, or will develop PTB.

The protein “E-cadherin” (E-CAD) is one of the key proteins in the tight junction (TJ) pathway. E-cadherin is a member of the Cadherin family which are calcium dependent adhesion molecules. Cadherins are a class of type I transmembrane proteins which play a critical role in cell adhesion ensuring that cells within tissues are bound together. E-cadherin consists of 5 cadherin repeats in the extracellular domain, one transmembrane domain, and an intracellular domain that binds catenin—thus linking the tight junction (TJ) and adherens junction (AJ) pathways. The sequence of human E-CAD is known; the sequence of human E-CAD preproprotein can be found at NCBI Reference Sequence: NP_{—}004351.1. This sequence is hereby expressly incorporated by reference.

Loss of E-cadherin function or expression has been implicated in cancer progression. Similarly, down-regulation of E-cadherin decreases the strength of cellular adhesion within a tissue, resulting in cellular motility. Allelic variants in E-cadherin are associated with a change in E-cadherin and have been demonstrated to confer increased risk for select disease states such as Crohn's disease.^{22, 23} While the data is very limited, recent reports suggest that polymorphisms in the E-cadherin promoter have been demonstrated to alter E-cadherin expression.²⁴ While this data argue that allelic variants in the AJ pathway are associated with both disease states and specific expression of AJ proteins, there is no data on this pathway or allelic variants in this pathway in obstetrics.

“Soluble E-cadherin” (SE-CAD) as used herein refers to the protein which is released when cadherin is spliced, indicating changes in adherens junction function. SE-CAD is an ˜80-kDa peptide degradation product of the 120-kDa E-cadherin molecule which is generated by a calcium ion dependent proteolytic process. Matrix metalloproteinases, trypsin, kallikrein 7, and plasmin are examples of molecules that are capable of performing this proteolytic process. As discovered by the inventor, SE-CAD is a marker of E-cadherin breakdown specifically in the cervix. SE-CAD can be measured in tissues and biological fluids; levels of SE-CAD indicate alterations in cadherin expression. The protein sequence of human SE-CAD corresponds to aa 1-750 of NCBI Reference Sequence: NP_{—}004351.1. This sequence is hereby incorporated by reference. In certain embodiments, “SE-CAD” as used in these methods is a smaller peptide than the 80 kDa peptide, or a fragment thereof. In certain embodiments, such fragments are those that are recognized by E-CAD antibodies that bind within the SE-CAD sequence, such as the antibody used in the R&D Biosystems ELISA described in the examples or other antibodies that recognize the SE-CAD sequence or a fragment specifically. Suitable SE-CAD fragments include at least 8-15 consecutive amino acids of the SE-CAD sequence. In other embodiments, such fragments include up to 25 aa, up to 50 aa, up to 75 aa, up to 100 aa, up to 150 aa, up to 200 aa, up to 300 aa, up to 400 aa, up to 500 aa, up to 600 aa, up to 700 aa, up to 750 consecutive amino acids of that sequence.

“Biological sample” or “sample” as used herein means any biological fluid or tissue that contains soluble E-cadherin (SE-CAD). The most suitable sample for use in the methods described herein includes urine, cervicovaginal fluid (CVF) and serum. Other useful biological samples include, without limitation, whole blood, plasma, saliva, vaginal mucus, cervical mucus, placental fluid, saliva, placental cells or tissue, cells or tissue of the cervix, and cells or tissue of the vaginal wall. In some examples, “blood” may refer to any blood component used as a sample such as whole blood, plasma or serum. Such samples may further be diluted with saline, buffer or a physiologically acceptable diluent. Alternatively, such samples are concentrated by conventional means.

By the terms “patient” or “subject” as used herein is meant a female mammalian animal, including a human, a veterinary or farm animal, a domestic animal or pet, and animals normally used for clinical research, including non-human primates, dogs and mice. More specifically, the subject of these methods is a human. In one aspect of the methods described herein, the subject undergoing the diagnostic or therapeutic method is asymptomatic for pre-term birth. In another aspect, the subject undergoing the diagnostic or therapeutic methods described herein shows clinical symptoms, or history, of pre-term birth.

“Clinical indicators of pre-term birth” as used herein, include, but are not limited to, prior PTB, short cervical length, bacterial vaginosis, maternal/uterine infection or inflammation, smoking, sexually transmitted diseases, African American race, low socio-economic status, stress, and depression.

“Healthy subjects” or “healthy control” as used herein refer to a subject or population of multiple subjects that did not develop pre-term birth. In one embodiment, healthy subjects may be a subject or population of multiple subjects that had pre-term labor, but did not develop PTB. In another embodiment, healthy subjects may be a subject or population of multiple subjects that never developed PTL or PTB.

“Same time of pregnancy” as used herein, means that the sample was collected when the control subject was in the same gestational week of pregnancy as the test subject. In one example, the control subject may be one gestational week earlier or later than the test subject.

“Short cervix” or “short cervical length” (CL) as used herein means a cervical length, as measured by transvaginal ultrasound, of 25 mm or less. in one embodiment, a short CL has a measurement of 15 mm or less.

“Fetal fibronectin” (FFN) as used herein refers to the protein produced by fetal cells. FFN is found at the interface of the chorion and the decidua (between the fetal sack and the uterine lining). A positive FFN test refers to the presence of fetal fibronectin in the subject's vagina, i.e., in the cervicovaginal fluid. A positive fetal fibronectin (FFN) test is strongly associated with PTB while a negative test is a strong predictor of the pregnancy continuing for at least 14 more days.

As used herein, the term “predetermined control” refers to a numerical level, average, mean or average range of the expression of a biomarker in a defined population. The predetermined control level is preferably provided by using the same assay technique as is used for measurement of the subject's SE-CAD levels, to avoid any error in standardization. For example, the control may comprise a single healthy pregnant mammalian subject at the same time of pregnancy as the subject. In another embodiment, the control comprises a single healthy pregnant mammalian subject who did not develop pre-term birth. In another embodiment, the control comprises a single healthy pregnant mammalian subject who had PTL, but did not develop PTB. In another embodiment, the control comprises a population of multiple healthy pregnant mammalian subjects at the same time of pregnancy as the subject or multiple healthy pregnant mammalian subjects who did not develop pre-term birth. In another embodiment, the control comprises a population of multiple healthy pregnant mammalian subjects at the same time of pregnancy as the subject or multiple healthy pregnant mammalian subjects who had pre-term labor but did not develop pre-term birth. In another embodiment, the control comprises the same subject at an earlier time in the pregnancy. In yet another embodiment, the control comprises one or multiple subjects with one or more clinical indicators of PTB, but who did not develop PTB. In addition, a predetermined control may also be a negative predetermined control. In one embodiment, a negative predetermined control comprises one or multiple subjects who had PTB.

As used herein, the term “control expression profile” refers to a numerical average, mean or average range of the expression of one or more biomarkers, including SE-CAD, in a defined population, rather than a single subject. For example, a positive control expression profile for one or more biomarker(s) in a healthy subject is a numerical value or range for expression of that biomarker(s) in a population of average healthy subjects who did not develop pre-term birth. Likewise, a negative control expression profile for the expression of one or more biomarker(s) in a subject with pre-term birth is a numerical value or range for the average expression of that biomarker(s) in a population composed of multiple patients who developed pre-term birth.

Unless defined otherwise in this specification, technical and scientific terms used herein have the same meaning as commonly understood by one of ordinary skill in the art to which this invention belongs and by reference to published texts.

It should be understood that while various embodiments in the specification are presented using “comprising” language, under various circumstances, a related embodiment is also be described using “consisting of” or “consisting essentially of” language. It is to be noted that the term “a” or “an”, refers to one or more, for example, “an immunoglobulin molecule,” is understood to represent one or more immunoglobulin molecules. As such, the terms “a” (or “an”), “one or more,” and “at least one” is used interchangeably herein.

### B. Diagnostic/Prognostic Methods for Pre-term Birth

In one embodiment, the invention provides a method for diagnosing, or predicting the likelihood of occurrence of, pre-term birth (PTB). The method comprises measuring the level of expression of a biomarker, soluble E-cadherin (SE-CAD), in a biological sample from a pregnant mammalian subject, wherein a level of expression of SE-CAD significantly different from the level of expression in a predetermined control is an indication of a diagnosis or likelihood of occurrence of pre-term birth. In another embodiment according to this method, the predetermined control is a level of SE-CAD in a control biological sample. In one embodiment, the control sample may be obtained from a healthy pregnant mammal at the same time of pregnancy as the subject. In another embodiment the control sample may be obtained from a healthy pregnant mammal who did not develop pre-term birth. In another embodiment, the control sample may be obtained from a healthy pregnant mammal who had PTL, but did not develop PTB. In a further embodiment, the control sample may be obtained come from a population of multiple healthy subjects described above. In another embodiment, the control sample may be obtained from the same subject at an earlier time in the pregnancy. In another embodiment of the invention, the level of expression of the biological sample or predetermined control is a mean or average, a numerical mean or range of numerical means, a numerical pattern, a graphical pattern or an expression profile.

In one embodiment, the biological sample is any sample as defined above. In another embodiment the biological sample is one, or more of serum, urine, and cervicovaginal fluid. In another embodiment, as is described, e.g., in Example 5 below, the biological sample is maternal serum. In another embodiment, the biological sample may be cervicovaginal fluid or urine. Example 7, below, describes the use of various biological fluids in the diagnostic methods described herein.

In one embodiment, these methods provide an indication of the likelihood of occurrence of PTB. In the preliminary studies described in the Examples below, for one cohort of women, mean levels of SE-CAD in maternal serum were significantly increased in women with PTB compared to those who had PTL but did not develop PTB. The odds of having PTB was 5.7 increased for women with SE-CAD levels above or equal to the mean level than for those below mean. These data support that PTB by itself functions as a viable biomarker in predicting PTB. Further, these results suggest that SE-CAD represents a biological process that is more intimately tied to the pathogenesis of PTB than race or even prior obstetrical history. Thus, in one embodiment, an expression level of SE-CAD in a subject greater than that of the healthy control indicates a 5-6 fold increase in the likelihood of developing PTB. In another embodiment, an increase of 10 pg/ml in the expression level of SE-CAD in a subject over that of a healthy control indicates a 200% increase in the likelihood of developing PTB. While these specific numerical indicators may be refined as commercial-scale data emerges, the correlation of SE-CAD with PTB, as described herein, is the critical diagnostic factor, i.e., a SE-CAD expression level over that of a healthy control is a positive indicator of an increased likelihood of occurrence of PTB.

In another embodiment, the method of the invention further comprises measuring the level of expression of at least one additional biomarker of PTB in the sample, wherein the combined changes in expression of SE-CAD and the additional biomarker from their respective levels of expression in the predetermined control is an indication of a diagnosis of PTB. In one embodiment, the additional PTB biomarker is fetal fibronectin in cervicovaginal fluid.

In another embodiment, the diagnostic method involves correlating an SE-CAD measurement with one or more clinical indicators of PTB selected amongst those described above in the mammalian subject providing the biological sample. Each of these clinical indicators provides additional data to the skilled clinician which may help provide a diagnosis, or indication of increased likelihood of, PTB. In one embodiment, the predetermined control may be tailored to account for the clinical indicators of PTB. For example, in an African American patient, a predetermined control comprising healthy African American subjects who did not develop PTB may be used. For example, the mean level of a predetermined control comprising all subjects with a clinical indicator of PTB, i.e., all African American subjects, all smokers, all women with prior PTB, may vary based on the clinical indicator chosen.

In still a further embodiment of methods according to this invention, a differential diagnosis of PTB in a subject may be performed by measuring SE-CAD levels in combination with measuring one of more second or other PTB biomarkers and/or coupled with clinical indicators of pre-term birth as described above. In one aspect, the diagnostic or disease monitoring methods described herein further include coupling the measurement of the biomarker SE-CAD with measuring the level of expression of at least one additional biomarker characteristic of PTB in a patient's sample. The combined changes in expression of SE-CAD and the additional biomarker from their respective levels of expression in a healthy mammalian subject is an indication of a differential diagnosis of increased likelihood of pre-term birth.

In another embodiment of the invention, the diagnostic method is performed on a subject's and/or predetermined control's biological samples obtained at or after 16 weeks of pregnancy. In one embodiment, the subject's/control's biological sample is obtained at or prior to 34 weeks of pregnancy. In yet another embodiment, the biological sample is obtained at 16-20 weeks of pregnancy. In another embodiment, the biological sample is obtained at 20-24 weeks of pregnancy. In another embodiment, the biological sample is obtained at 24-28 weeks of pregnancy.

Similarly, a method for monitoring progression of PTB in a mammalian subject involves measuring the level of expression of SE-CAD in a biological sample from a mammalian subject having PTB over a given time period. In one embodiment, the method of the invention further comprises repeating the measurement of SE-CAD levels multiple times during the subject's pregnancy. In one embodiment, the measurement is repeated two times during the pregnancy. In another embodiment, the measurement is repeated three, four, five or more times during the pregnancy. In another embodiment, the method of the invention further comprises measuring SE-CAD levels in a series of subject samples taken at different times during the pregnancy and identifying a pattern of increased expression of SE-CAD throughout the pregnancy. In one embodiment a suitable time period includes a baseline at about 16 weeks, with repeated SE-CAD measurements every 6-8 weeks thereafter. In other embodiments, the subsequent SE-CAD measurements are repeated more frequently after 32 weeks of pregnancy where the trend of SE-CAD levels appears higher as progressively measured. In still other embodiments, a physician may select a different appropriate assessment period.

In another embodiment, the subject is being treated for PTL or increased likelihood of PTB and wherein the method enables a determination of the efficacy of the treatment. In one embodiment, the method involves measuring the level of expression of SE-CAD in a biological sample from a mammalian subject having PTL over a given time period. The expression level of SE-CAD is then compared with the level of expression in one or more biological samples of the same subject assayed earlier in time, or before or during treatment. In one embodiment the comparison can occur by direct comparison with one or more prior assessments of the same patient's status. In another embodiment, the reference may be a negative control comprising subjects with PTB. In another embodiment, the same or decreased expression level of the SE-CAD in a biological sample of the subject compared to that of an earlier biological sample of the same subject or predetermined control is indicative of the efficacy of the treatment. A level of expression of the SE-CAD in the subject's sample that is above the subject's prior level of expression (or reference average) is an indication of progression of PTL, or lack of efficacy of the treatment. In another embodiment, a decrease in the rate of increased expression of SE-CAD expression over the course of treatment is indicative of the efficacy of treatment.

In yet another embodiment, the diagnostic method involves measuring the expression of SE-CAD as nucleic acid, e.g., mRNA, DNA, cDNA, or as a protein. In another embodiment, the method further includes contacting the biological sample from a subject with a diagnostic reagent that measures a first level of SE-CAD nucleic acid or protein in the sample. In yet another embodiment, the method involves contacting a second biological sample from the subject at a second later time during the pregnancy and measuring a second level of SE-CAD. A diagnosis of increased risk of pre-term birth may be provided based upon an increase in the second level over that of a healthy pregnant mammalian subject at the same time of pregnancy as the second sample from the subject. In another embodiment, the diagnosis may be provided based on an increase in the second level over a healthy pregnant mammalian subject who did not develop pre-term birth. In another embodiment, the diagnosis may be provided based on an increase in the second level over a population of multiple control subjects as described above. In another embodiment, the diagnosis may be provided based on an increase in the second level over the level of SE-CAD of the same subject at an earlier time in the pregnancy. In one embodiment, the subject has an increased risk of developing pre-term birth if the second SE-CAD sample level is higher than any of the reference levels described above.

In another embodiment, the contacting step comprises forming a direct or indirect complex in the subject's biological sample between a diagnostic reagent for SE-CAD and the SE-CAD nucleic acid or protein in the sample. In yet another embodiment, the contacting step further comprises measuring a level of the complex in a suitable assay. In one embodiment, as is utilized in the examples described below, the assay is an enzyme-linked immunosorbent assay (ELISA). See, the examples below, wherein the R&D BIOSYSTEMS human E-CAD monoclonal antibody ELISA assay kit was used.

In one embodiment, the antibody specifically binds to at least part of, i.e., a fragment or epitope of the 750 aa SE-CAD sequence. Such fragments or epitopes include 8-15 amino acids, up to 25 aa, up to 50 aa, up to 75 aa, up to 100 aa, up to 150 aa, up to 200 aa, up to 300 aa, up to 400 aa, up to 500 aa, up to 600 aa, up to 700 aa, up to 750 aa. For example, one commercially available antibody from Lifespan Biosciences, binds to the sequence between 600-707 aa of human SE-CAD. It is within the skill of the art to select an antibody that specifically binds an appropriate epitope of the 750 aa SE-CAD sequence within the 750 aa SE-CAD sequence.

In another embodiment, the suitable assay is selected from the group consisting of an immunohistochemical assay, a counter immuno-electrophoresis, a radioimmunoassay, radioimmunoprecipitation assay, a dot blot assay, an inhibition of competition assay, and a sandwich assay. In another embodiment, the diagnostic reagent is labeled with a detectable label. In one embodiment, the label is an enzyme, a fluorochrome, a luminescent or chemi-luminescent material, or a radioactive material. In another embodiment, the diagnostic reagent is an antibody or fragment thereof specific for SE-CAD. In another embodiment, the diagnostic reagent is a polynucleotide or genomic probe that hybridizes to SE-CAD cDNA or mRNA. Such polynucleotides may be about 25 or more nucleotides in length. In another embodiment, the diagnostic reagent is a PCR primer-probe set that amplifies and detects a polynucleotide sequence of SE-CAD mRNA. In one embodiment, the reagent is immobilized on a substrate. In another embodiment, the diagnostic reagent comprises a microarray, a microfluidics card, a computer-readable chip or chamber. In another embodiment, the diagnostic reagent enables detection of changes in expression in SE-CAD in the subject's biological sample from that of a reference expression profile, the changes correlated with the likelihood of PTB.

In yet another embodiment of the method of the invention, the measuring is performed by a computer processor or computer-programmed instrument that generates numerical or graphical data useful in diagnosing the likelihood of PTB. In one embodiment, the method of the invention further comprises coupling the relationship of the sample SE-CAD level with the predetermined control level and further with the presentation of clinical indicators of PTB in the subject described above. In another embodiment, the method further comprises coupling the comparative relationship of the sample SE-CAD level with the predetermined control with a history of pre-term birth. In another embodiment, the method provides a quantitative assessment of the likelihood or risk of pre-term birth in a subject who has not yet developed clinical symptoms of pre-term birth.

In another embodiment, the invention provides a method for screening a population of pregnant women for premature cervical remodeling comprising measuring the level of expression of a biomarker, soluble E-cadherin (SE-CAD), in a biological sample from a pregnant mammalian subject, wherein an increased level of expression of SE-CAD above the level of expression in a predetermined healthy control is an indication of premature cervical remodeling.

The levels of SE-CAD in a population can fluctuate based upon multiple variables. For example, as described in the examples below, in one experiment, SE-CAD levels in CVF were much lower than in maternal serum but were detectable in all samples. In addition variations in an individual assay used for measurement and the standardization of regents employed in such assay may provide variations measured SE-CAD levels. Therefore, in one embodiment of this invention, i.e., that based upon the sample, assay and antibody employed in the examples below, the level of serum SE-CAD ranges between and including the concentrations 2-65 pg/ml. For example, according to the ELISA assay employed in the examples below, mean levels of SE-CAD levels in maternal serum for control patients who did not develop PTB were 47.4 pg/ml, while average serum SE-CAD concentration in patients who developed PTB was 58.8±7.1 pg/ml (Example 4). Thus, in one embodiment, a serum SE-CAD level above 47.4 is indicative of a diagnosis of increased likelihood of PTB. In an example described below, average levels of SE-CAD in maternal urine were about 21.8 pg/ml. Thus, in one embodiment, a urine SE-CAD level above 21.8 pg/ml is indicative of a diagnosis of increased likelihood of PTB. However, as described above, these specific values are by no means limiting, as other averages may be obtained for larger populations or populations of patients differing in other physiological characteristics, e.g., clinical symptoms of PTB, weight, etc. or for other types of biological samples.

In one embodiment of the method of this invention, when using an ELISA, particularly the ELISA and reagents of the examples below, a diagnosis of PTB or increased likelihood of PTB may be obtained when a subject's serum SE-CAD is elevated above 40 pg/ml. In another embodiment, a diagnosis of PTB may be made when a subject's serum SE-CAD level is greater than 30 pg/ml. In yet another embodiment, a diagnosis of PTB may be made when a subject's serum SE-CAD level is or greater than 35 pg/ml. In yet another embodiment, a diagnosis of PTB may be made when a subject's serum SE-CAD level is or greater than 45 pg/ml. In yet another embodiment of the diagnostic method of this invention a diagnosis of PTB may be made when a subject's serum SE-CAD level is or greater than 50 pg/ml, and so on. In another embodiment, diagnosis of PTB may be made when a subject's SE-CAD level in a urine sample is greater than 21.8 +/−5 pg/ml. In another embodiment, diagnosis of PTB may be made when a subject's SE-CAD level in a urine sample is greater than 30 pg/ml. In another embodiment, diagnosis of PTB may be made when a subject's SE-CAD level in a urine sample is greater than 35 pg/ml. In another embodiment, diagnosis of PTB may be made when a subject's SE-CAD level in a urine sample is greater than 40 pg/ml.

As stated above, specific diagnostic methodology employed in the measurement of SE-CAD in the biological sample includes measuring the SE-CAD as ribonucleic acid (mRNA, i.e., measuring the transcription of SE-CAD) or protein (i.e., measuring translation of the protein) using conventional assay technologies. In one embodiment, the SE-CAD expression is measured in the urine, cervicovaginal fluid or blood at the mRNA and protein levels, respectively by polymerase chain reaction and enzyme-linked immunosorbent assay (ELISA). The specific methodologies that can be employed to perform the diagnostic methods described herein are conventional and may be readily selected and adapted by one of skill in the art, including the exemplified ELISA assay for SE-CAD in a urine, blood or cervicovaginal fluid sample.

1. Assays for Protein

The measurement of the SE-CAD protein (or any second biomarker) in the biological sample may employ any suitable SE-CAD ligand, e.g., antibody (or antibody to any second biomarker) to detect the protein. Such antibodies may be presently extant in the art or presently used commercially, such as those available as part of the R&D BIOSYSTEMS human E-CAD monoclonal antibody ELISA assay kit or may be developed by techniques now common in the field of immunology. As used herein, the term “antibody” refers to an intact immunoglobulin having two light and two heavy chains or any fragments thereof. Thus a single isolated antibody or fragment may be a polyclonal antibody, a high affinity polyclonal antibody, a monoclonal antibody, a synthetic antibody, a recombinant antibody, a chimeric antibody, a humanized antibody, or a human antibody. The term “antibody fragment” refers to less than an intact antibody structure, including, without limitation, an isolated single antibody chain, a single chain Fv construct, a Fab construct, a light chain variable or complementarity determining region (CDR) sequence, etc. A recombinant molecule bearing the binding portion of an SE-CAD antibody, e.g., carrying one or more variable chain CDR sequences that bind SE-CAD, may also be used in a diagnostic assay of this invention. As used herein, the term “antibody” may also refer, where appropriate, to a mixture of different antibodies or antibody fragments that bind to SE-CAD. Such different antibodies may bind to a different portion of the SE-CAD protein than the other antibodies in the mixture. Such differences in antibodies used in the assay may be reflected in the CDR sequences of the variable regions of the antibodies. Such differences may also be generated by the antibody backbone, for example, if the antibody itself is a non-human antibody containing a human CDR sequence, or a chimeric antibody or some other recombinant antibody fragment containing sequences from a non-human source. Antibodies or fragments useful in the method of this invention may be generated synthetically or recombinantly, using conventional techniques or may be isolated and purified from plasma or further manipulated to increase the binding affinity thereof. It should be understood that any antibody, antibody fragment, or mixture thereof that binds SE-CAD or a particular sequence of SE-CAD as defined above may be employed in the methods of the present invention, regardless of how the antibody or mixture of antibodies was generated.

Similarly, the antibodies may be tagged or labeled with reagents capable of providing a detectable signal, depending upon the assay format employed. Such labels are capable, alone or in concert with other compositions or compounds, of providing a detectable signal. Where more than one antibody is employed in a diagnostic method, e.g., such as in a sandwich ELISA, the labels are desirably interactive to produce a detectable signal. Most desirably, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength.

Other label systems that may be utilized in the methods of this invention are detectable by other means, e.g., colored latex microparticles (Bangs Laboratories, Indiana) in which a dye is embedded may be used in place of enzymes to provide a visual signal indicative of the presence of the resulting SE-CAD-antibody complex in applicable assays. Still other labels include fluorescent compounds, radioactive compounds or elements. Preferably, an anti-SE-CAD antibody is associated with, or conjugated to a fluorescent detectable fluorochromes, e.g., fluorescein isothiocyanate (FITC), phycoerythrin (PE), allophycocyanin (APC), coriphosphine-O (CPO) or tandem dyes, PE-cyanin-5 (PC5), and PE-Texas Red (ECD). Commonly used fluorochromes include fluorescein isothiocyanate (FITC), phycoerythrin (PE), allophycocyanin (APC), and also include the tandem dyes, PE-cyanin-5 (PC5), PE-cyanin-7 (PC7), PE-cyanin-5.5, PE-Texas Red (ECD), rhodamine, PerCP, fluorescein isothiocyanate (FITC) and Alexa dyes. Combinations of such labels, such as Texas Red and rhodamine, FITC +PE, FITC+PECy5 and PE+PECy7, among others may be used depending upon assay method.

Detectable labels for attachment to antibodies useful in diagnostic assays of this invention may be easily selected from among numerous compositions known and readily available to one skilled in the art of diagnostic assays. The SE-CAD antibodies or fragments useful in this invention are not limited by the particular detectable label or label system employed. Thus, selection and/or generation of suitable SE-CAD antibodies with optional labels for use in this invention is within the skill of the art, provided with this specification, the documents incorporated herein, and the conventional teachings of immunology.

Similarly the particular assay format used to measure the SE-CAD in a biological sample may be selected from among a wide range of immunoassays, such as enzyme-linked immunoassays, such as those described in the examples below, sandwich immunoassays, homogeneous assays, immunohistochemistry formats, or other conventional assay formats. One of skill in the art may readily select from any number of conventional immunoassay formats to perform this invention.

Other reagents for the detection of protein in biological samples, such as peptide mimetics, synthetic chemical compounds capable of detecting SE-CAD may be used in other assay formats for the quantitative detection of SE-CAD protein in biological samples, such as high pressure liquid chromatography (HPLC), immunohistochemistry, etc.

2. Nucleic Acid Assay Formats

Still other methods useful in performing the diagnostic steps described herein are known in the art. Such methods include methods based on hybridization analysis of polynucleotides, methods based on sequencing of polynucleotides, proteomics-based methods or immunochemistry techniques. The most commonly used methods known in the art for the quantification of mRNA expression in a sample include northern blotting and in situ hybridization; RNAse protection assays; and PCR-based methods, such as reverse transcription polymerase chain reaction (RT-PCR) or qPCR. Alternatively, antibodies may be employed that can recognize specific DNA-protein duplexes. The methods described herein are not limited by the particular techniques selected to perform them. Exemplary commercial products for generation of reagents or performance of assays include TRI-REAGENT, Qiagen RNeasy mini-columns, MASTERPURE Complete DNA and RNA Purification Kit (EPICENTRE®, Madison, Wis.), Paraffin Block RNA Isolation Kit (Ambion, Inc.) and RNA Stat-60 (Tel-Test), the MassARRAY-based method (Sequenom, Inc., San Diego, Calif.), differential display, amplified fragment length polymorphism (iAFLP), and BeadArray™ technology (Illumina, San Diego, Calif.) using the commercially available Luminex100 LabMAP system and multiple color-coded microspheres (Luminex Corp., Austin, Tex.) and high coverage expression profiling (HiCEP) analysis.

### C. EXAMPLES

The examples that follow do not limit the scope of the embodiments described herein. One skilled in the art will appreciate that modifications can be made in the following examples which are intended to be encompassed by the spirit and scope of the invention.

### Example 1

### Preterm Parturition from Intrauterine Inflammation Results in Decreased Expression of E-cadherin in Cervical Tissues

In the examples herein, a mouse model of intrauterine inflammation¹⁶⁻¹⁸ was used in which lipopolysaccharide (a component of the cell wall of gram negative bacteria, LPS) is infused into the uterine horn. Dams deliver between 8-20 hours, with most delivering in 12 hours with no maternal mortality.^{14, 15, 19-21} Intrauterine inflammation is associated with premature cervical ripening that occurs prior to the clinical onset of parturition.¹⁶ Compared to controls, cervices from dams exposed to intrauterine LPS, had fewer collagen fibers, a less dense stroma, and an increase in mucin.¹⁶ Cervical ripening appears to be the initial event in the pathogenesis of inflammation-induced PTB.

The mouse model of intrauterine inflammation described above was employed and cervical tissues were harvested from dams exposed to LPS or saline at 2, 4 and 6 hours. Using QPCR, it was demonstrated that mRNA expression of E-cadherin in the cervix is not altered at 2 or 4 hours after exposure to intrauterine inflammation but expression is decreased 2.7 fold (P=<0.05) in the cervix at 6 hours in LPS exposed dams compared to saline-exposed.

### Example 2

### Term Parturition is also Associated with a Decrease in E-Cadherin mrna Expression as this Pathway in Cervical Remodeling is Hypothesized to be Common to all Parturition

E-cadherin mRNA expression was measured with QPCR in non-pregnant cervices, across gestation in timed-pregnant mice as well as postpartum. For this strain of mice, parturition occurs in a predictable time course on E19. “E#” refers to Embyonic Day in defining the gestation period of mice, where the first 24 hours of gestation are counted as E0. Therefore, the 12 hours prior to E19 (E18.5) represent the time just prior to term parturition. E-cadherin mRNA levels were significantly different between the groups (P=0.001, One Way ANOVA). Compared to non-pregnant levels, E-cadherin mRNA was increased on E15 (preterm; mid-gestation)(P=0.007, SNK). E-cadherin mRNA levels are decreased 4.3 fold on E18.5 compared to E15 (P=<0.001, SNK), which supports that expression of E-cadherin also occurs with term parturition,.

### Example 3

### SE-Cadherin (SE-CAD) is Increased in Maternal Serum Prior to Delivery in a Mouse Model of Inflammation-Induced Preterm Birth

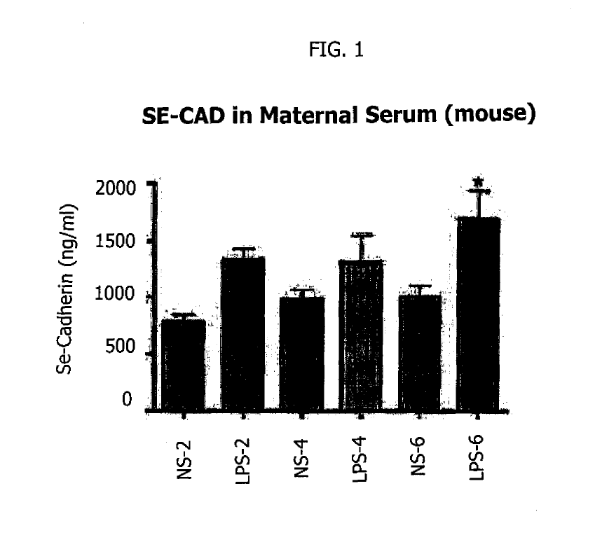

SE-CAD was measured in maternal serum at 2, 4 and 6 hours after intrauterine infusion of LPS or saline using the mouse model of Example 1. SE-CAD was measured using a commercially available ELISA (R&D Biosystems human E-CAD monoclonal antibody ELISA kit). As noted in FIG. 1, SE-CAD levels are significantly different between LPS and saline exposed dams (P=<0.001, One-Way ANOVA). SE-CAD levels continue to rise at six hours (prior to the clinical onset of parturition in this model). These data support that SE-CAD in the serum is a marker of E-cadherin break down specifically in the cervix, and that cervical remodeling can be detected systemically.

### Example 4

### SE-CAD Levels in Human Maternal Serum Predict Preterm Birth

SE-CAD levels were examined in maternal human serum and the ability of SE-CAD to accurately predict PTB was evaluated to determine if SE-CAD levels can be detected systemically and are a biomarker of cervical remodeling. A prospective cohort of women with preterm labor (PTL) (n=110) was used. Singleton pregnancies between 22-34 weeks of gestation were eligible. Maternal serum was obtained at time of presentation of PTL. Gestational age at delivery and maternal and neonatal outcomes were collected.

SE-CAD levels were assessed in these serum samples by the same commercially available ELISA as used in Example 3 (R&D Biosystems). Unadjusted analyses and multivariable logistic regression were performed. For this cohort, 50% delivered at <37 wks. Mean levels of SE-CAD levels in maternal serum were significantly increased in women with PTB (58.8+/−7.1) compared to those without (47.4+/−190.6) (P<0.0001). Controlling for prior PTB, cervical exam at presentation and race, it was determined that for each 10 units of SE-CAD, the odds of PTB increased by 202% (CI 1.3-3.1). In an ROC analysis, the AUC (area under the curve) for SE-CAD alone was 0.73 and was not altered when considering other covariates. The odds of having PTB is 5.7 increased for women with SE-CAD above or equal to mean (46.1 +/−12.7) than those below (2.2−14.5, p=0.0002). At this level, the positive predictive value (PPV) is 76% and the negative predictive value (NPV) 70% for PTB.

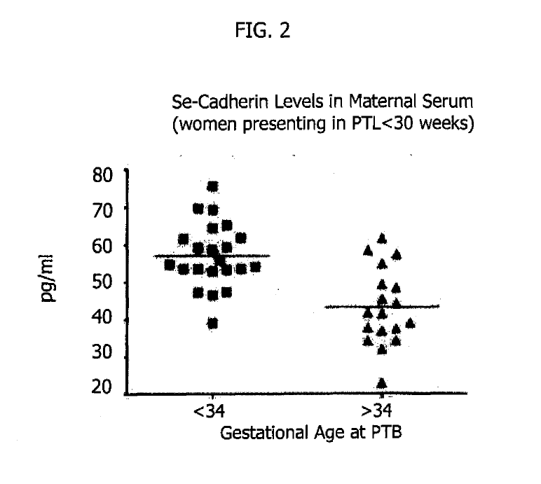

When investigating those women who presented with PTL at <30 weeks, SE-CAD levels were even more discriminatory and predicted those women who eventually delivered at <34 weeks (see FIG. 2). These data demonstrate that SE-CAD alone appears to function well in predicting PTB. These results suggest that SE-CAD represents a biological process that is more intimately tied to the pathogenesis of PTB than race or even prior obstetrical history.

Current strategies to predict PTB, such as FFN or cervical length, are problematic in that they have either a low positive predictive value or/and poor sensitivity. This is in contrast to SE-CAD, which in the preliminary data, has PPV of 76% while still conferring a 70% NPV.

### Example 5

### SE-CAD Levels are Detectable in Diverse Biological Fluids and can be Utilized to Predict Risk of PTB Prior to Clinical Onset of PTL or PTB

To determine if SE-CAD levels from earlier in gestation (16-20 wks) were detectable and associated with PTB in asymptomatic women, SE-CAD levels were assessed in cervicovaginal fluid (CVF; n=59) collected at 16-20 weeks in another prospective cohort of low risk women. Results of the ELISA demonstrated that SE-CAD levels in CVF were much lower than in maternal serum but were detectable in all samples. In CVF, there was a significant interaction between SE-CAD levels and bacterial vaginosis (BV) (P=0.04). BV was diagnosed by Nugent's criteria. In women without BV, increasing levels of SE-CAD were associated with a 3.2-increased odds of PTB.

In addition to measuring SE-CAD in CVF, SE-CAD was also measured in urine samples from a prospective cohort of low-risk women as part of a study to assess biomarkers of preeclampsia. Women with delivery at <35 weeks were excluded from follow-up. Thus, only the outcome of late PTB (35-36 0/6 week) was available to correlate SE-CAD levels.

The urine was collected from patients at 16-20 weeks. SE-CAD levels were measured using the ELISA identified above and were detectable in maternal urine in the 2nd trimester. Urinary levels of SE-CAD were between those measured in CVF and serum, based on mean sample values. The mean SE-CAD levels in urine of women destined to deliver preterm (n=20) was 31.5 +/−6, while mean SE-CAD urine levels in women who ultimately had a term delivery (n=19) was 21.8 +/31 5. These differences were not statistically significant, but this is believed to be due to the limited sample size. The ‘phenotype’ of the PTB outcome was less severe than in the two experiments reported above which investigated subjects with PTB occurring >34 weeks.

### Example 6

### SE-CAD Levels in Women with Preeclampsia

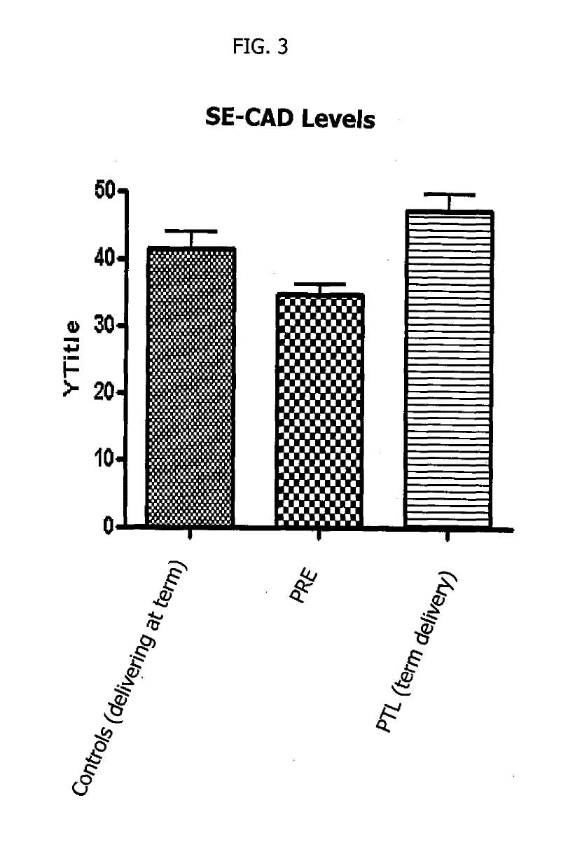

In the study referred to above, SE-CAD levels were not different in women with preeclampsia compared with controls. As shown by the results in FIG. 3, SE-CAD levels in women delivering at term are about the same as women from the preterm labor group—who did not have preterm delivery. The controls (first column) were women at term—some of which were in labor. This is in contrast to women with preeclampsia—none who were in labor—who actually had lower SE-CAD levels. This further gives credibility that SE-CAD is from the cervix and associated with labor. The fact that women with preterm labor have the same levels as those women delivering at term may indicate some level of cervical change in women with preterm labor even without eventual preterm delivery. Also, baseline levels, obtained earlier in pregnancy, in women without symptoms, may be lower than the cutoff of this study, i.e., ˜40 pg/ml. If so, then higher levels of SE-CAD in women who deliver preterm are likely to be of even higher predictive value. These data further show that increased SE-CAD levels are indicative of cervical remodeling and a novel predictor of preterm birth.

### Example 7

### Large Scale Human Studies

A prospective cohort of pregnant women is enrolled for a large scale study. Pregnant women are evaluated at three time points in pregnancy: 16-20 weeks, 20-24, and 24-28 weeks. At all three data collection time points, the following are collected unless otherwise noted: maternal DNA is collected at enrollment with Oragene collection tubes (collected at first visit only); an assessment of cervical length is made by transvaginal ultrasound and digital cervical examination. Maternal serum and cervicovaginal fluid (CVF) are collected for use for both biomarker analysis and to assess presence of bacterial vaginosis. Maternal urine is collected for screening for bacterial vaginosis.

Inclusion criteria are pregnant women with singleton gestations screened at <20 weeks at the first prenatal visit who agree to participate in the study. Women with any prior obstetrical history are included. Women with prior preterm birth are offered 17-alpha hydroxyprogesterone caproate (17OHPC) per clinical standard of care. Regardless of use of 17OHPC, these women with prior PTB are included. The use of 17OHPC, including the number of injections, is recorded.)

Exclusion criteria: Women with a multi-fetal pregnancy, chronic medical disease with significant end-organ injury (e.g. chronic hypertension with renal disease; transplant patients; women with major adult congenital heart disease), pregestational diabetes Class D or greater, current use of immunosuppressive therapy or enrollment for prenatal care after 20 weeks are not eligible for the study.

Enrollment: Pregnant women are approached for enrollment at <20 weeks during a routine obstetrical visit. Women agreeing to be in the study have a ‘research visit’ either at that same visit (if 16-20 weeks) or arranged at the same time as their next prenatal visit. The first data collection occurs at 16-20 weeks. Women are evaluated again between 20-24 and 24-28 weeks. This research visit coincides with their regular prenatal care visit when feasible. The interval between visits is documented for each patient.

Clinical Protocol: The collection of the data/biospecimens is obtained in a dedicated research room. The ultrasound, examination and specimen collection is performed by the research coordinators (research nurses) who are trained in the appropriate methods for specimen collection as well as trained for assessing cervical length by transvaginal ultrasound. A case report form (CRF) includes all variables listed below as well as the results of the data collection from all time points (e.g. SE-CAD levels, BV presence, cervical length, etc).

1. Transvaginal ultrasound for cervical length. The cervical length (CL) is imaged in three views and the shortest distance recorded. As the data regarding the ‘treatment’ options for ultrasonographic short cervix is unclear, the results of this study are not revealed to caring physicians unless a ‘critical value’ is obtained. A critical value is considered a CL of 15 mm or shorter based on recent studies.⁸ The expected prevalence of finding is about 1.5%;⁸ thus, we are likely to find short cervix in about 51 patients. Currently standard of care at our institution is that we do not routinely screen for CL. Yet, when a short cervix (15 mm or <) is incidentally found, the patients are counseled by the Maternal Fetal Medicine service regarding cerclage, the use of progestational agents, or no intervention. This counseling occurs independently of the study and not by the PI of the study. These patients are not excluded from continuing in this study. The purpose of measuring CL in this cohort is not to investigate its primary association with PTB as this has been well documented in large trials. Indeed, while a short cervix by ultrasonography is a strong risk factor for PTB, the low prevalence of a ‘very short cervix’ and poor sensitivity have limited the use of ultrasonography to detect short cervix as a reliable screening tool for PTB in all women.^{8, 9}

The purpose of measuring CL in this study is to assess the interaction of SE-CAD with CL with the hypothesis that they both represent different pathways involved in ‘cervical remodeling’. Less ‘critical values’ but still shortened CLs (e.g. 15-25 mm) are anticipated to have a stronger association with PTB when higher SE-CAD levels are present, indicating more active cervical remodeling. CL images are recorded and printed by the research nurse. The research nurses are trained by the PI who is a board-certified perinatalogist and proficient in transvaginal ultrasound. Each nurse is required to perform TV ultrasound imaging of the cervix on 10 patients, obtaining 3 images of the cervix. Those patients remain in the study but are excluded from any analyses on cervical length.

2. Cervical digital exam for length and dilatation. Cervical dilatation of the internal os is recorded and rounded up to nearest whole number (recorded as 0, 1, 2 etc). Considering the variability and subjectively in effacement and softening, these are not recorded. Cervical exam is being measured to determine the correlation of dilatation with SE-CAD levels to see if a clinical parameter equates with a biomarker of cervical remodeling. Cervical dilatation is used in our statistical modeling for a prediction model.

3. Bacterial Vaginosis (BV): Patients are screened for the presence of BV using Nugent's criteria²⁵ at this each data collection time point. The presence of BV has been associated with PTB and premature rupture of membranes, especially in high risk patients. Yet, BV alone is insufficient as a risk stratifying marker for PTB. Preliminary data suggests that the presence of BV, even in a low risk population, modifies the association of SE-CAD in CVF with PTB as well as modify the association of claudin SNPs with PTB. Preliminary studies suggest that BV is, in fact, a strong environmental modifier for PTB. For this study, enrolled patients are screened for BV at the three data collection time points; the gold standard for overall diagnosis of BV is made using the criteria developed by Nugent.²⁵ A provider collected vaginal swab is obtained and the swab material is spread on a glass slide (a unique bar code sticker is placed), air dried, and transported to the microbiology lab for gram staining. The vaginal smear is evaluated (according to Nugents criteria) for the following morphotypes: Lactobacillus sp.: straight gram-positive rods; Gardnerella or Bacteroides sp.: small gram -variable or small gram-negative bacilli; Mobiluncus sp.: curved gram-negative rods. Each of the above morphotypes is quantitated separately according to the average number of each per oil immersion field. If the total score is ≧7, the gram stain is consistent with BV. The score for each specimen (at each time point) is recorded and entered into the access database with all other variables.

4. Cervicovaginal fluid (CVF): Two swabs are placed in the cervicovaginal fornix and then placed in PBS and immediately placed in liquid nitrogen for future evaluation. The specimens are collected per established protocols of co-investigators on this proposal.²⁶ Collection of specimens is performed to allow not only for the assessment of SE-CAD but also for future protein studies (e.g. ELISAs).

5. Urine specimen: The urine specimen is spun down to remove any cellular debris and then immediately placed in liquid nitrogen. These specimens are used to assess SE-CAD levels.

6. Maternal serum: Maternal blood is drawn; the serum collected is immediately placed in liquid nitrogen. These specimens are used to assess SE-CAD.

7. Maternal DNA: At the first data collection time point, maternal DNA is collected using the Oragene.

8. Processing of Biological Specimens: Maternal blood, urine and CVF specimens are processed and then serum is aliquoted to 5 pre-labeled tubes (de-identified), placed in liquid nitrogen containers and then transported to the laboratory. ELISAs for SE-CAD are commercially available (R&D Biosystems). We have validated these ELISAs in our laboratory with all of the proposed biological specimens. ELISAS for various maternal immune biomarkers are also commercially available and have been validated and reported in the inventor's laboratory and the description thereof incorporated by reference.¹⁹Maternal DNA is collected using the Oragene system from maternal saliva. Saliva samples stabilized in Oragene kits provide a robust source of genomic DNA that can be stored and shipped under ambient or refrigerated conditions; samples are collected according to the manufacturer's instructions. DNA assays are conducted. An aliquot of each Oragene saliva sample is used for automated DNA extraction on the Chemagen system, typically producing more than 100 micrograms of genomic DNA. Unused DNA samples are archived at −80C with bar-coding and sample tracking in the Freezer DB database.

9. Clinical & other variables to be collected:

Maternal Demographics: Race, ethnicity, age; BMI at first prenatal visit and each data collection time point; smoking history, education level

Current Obstetrical history: presence of STD during this pregnancy; HSV in this pregnancy; pap smear result this pregnancy; use of progestational agents during this pregnancy, any episodes of preterm labor, use of tocolytics, administration of betamethasone for fetal indications, presence of any obstetrical complications including abruption, chorioamnionitis, preeclampsia, and/or fetal demise.

Past medical and surgical history; GYN history: history of STD, HSV, fibroids, abnormal pap smears; HPV; Social history including tobacco, alcohol and illicit drug use

Prior Obstetrical History: Number of prior pregnancies, number of term deliveries, number of preterm deliveries, gestational age at preterm delivery, birth weights, use of 17OHPC in any pregnancy, history of preeclampsia, number of spontaneous miscarriages, number of 2nd trimester spontaneous loss, history of fetal demise

Delivery information: mode of delivery, use of induction agents, use of antibiotics in labor, fever in labor, and reason for induction

Neonatal information: Birth weight, admission to NICU, presence of adverse neonatal outcomes at 7 or 30 days of life (IVH, NEC, etc). Data abstraction of these variables is conducted using an established method for obtaining the highest quality data from these charts.^{27, 28}

A short questionnaire about depressive symptoms (Edinburgh Postnatal Depression Scale, EPDS) is performed at enrollment. This instrument is a validated measure for assessing depressive symptoms during pregnancy.²⁹⁻³² Recent studies suggest that depression early in pregnancy as assessed by a similar instrument is an independent risk factor for PTB;⁵ however, this study has only 5% African-American patients and as such may not be relevant for the group of women with the highest rates of PTB. In our current prospective trial of women with preterm labor, EPDS has been completed on 95% of women enrolled. Using this instrument for the proposal herein will provide another environmental marker that may confer an independent risk for PTB but may also be a strong environmental modifier of genetic risk.

A questionnaire about health system distrust and experiences of discrimination (EOD) is self-administered at the first data collection time point.³³ There is a growing body of literature suggesting that perceived discrimination results in biological stress and that this stress is associated, if not causative in PTB.^{2, 3, 4, 34} These validated questionnaires were used in the current prospective trial of preterm labor patients. To date, of those enrolled, 99% have completed the questionnaire. This validated instrument is an assessment of perceived racism and is believed to reflect an environmental stress level.³³ For the EOD, analysis is performed as mean score as well as counts of experiences of discrimination as previously described.³³

10. Sample Size Assumptions

The prevalence of PTB in the whole cohort is assumed to be about 9% at <35 weeks and 12% at <37 weeks of gestation. This prevalence is based on recent multicenter randomized control trial based from our center and published data.³⁵ Based on prior and current prospective cohorts, enrollment for these type of studies is greater than 70%. However, based on the need for repeat visits, the enrollment is estimated to be at 45%. Approximately 75-80% of women enrolled in current and recent prospective trials are African American. Based on prior prospective studies, the percent of new obstetrical visits that were nulliparous women was 36%. A type 1, alpha error of 0.05 is assumed; a precision of +/−8.5; a loss to follow-up of 5%. A pregnancy loss rate of 10% is assumed (likely to be an over-estimation since enrollment is occurring after establishment of prenatal care and confirmation of fetal viability routinely occurs at first visit). A loss of 15% is assumed due to medical complications (based on exclusion criteria). With these assumptions, about 3391 patients are enrolled. Based on a prevalence of PTB of ˜12% at <37 weeks, this will provide 407 cases or patients who deliver at <37 weeks.

11. Descriptive Statistics

In this study, cases of PTB and non-cases (defined as those who deliver >37) are characterized by gestational age at admission, maternal age, race, obstetrical history, and other variables that are potential risk factors. Categorical variables are summarized by frequencies and proportions, and continuous variables are summarized by the mean, median, standard deviation, and range. Cases and noncases are described overall, and by phenotypes of PTB based on gestational ages. Based on previous work, estimates are approximately 7% PTB<35 weeks, 5% <32 weeks and 2% <28 weeks. These prevalences are evaluated with 0.9, 0.8 and 0.5% error, respectively. Additionally, sufficient power is used to detect odds ratios of at least 2.2 in the rarest phenotype (<28 weeks) and less than 2.0 in the other phenotypes in the bivariate analyses to follow.

Bivariate Analysis of Candidate Risk Factors: SE-CAD in each fluid compartment and other risk factors for PTD will first be analyzed as a separate risk factor for the outcome under study. Each of the three time points for SE-CAD collection is analyzed separately. Distributions on discrete variables are characterized by proportions and compared by chi-square or exact methods, as appropriate. Relative risks and 95% confidence intervals are calculated for dichotomous risk factors. The Student's t test (for approximately normally distributed data) or Mann Whitney U test (for ordinal or non-normally distributed variables) is used to compare cases and non-cases with respect to continuous variables. Stratified analyses are performed for the preliminary assessment of confounding and interaction. Of particular interest will by the odds ratios for nulliparous and multiparous women. While a separate analysis is planned for nulliparous women, if confounding is evident by parity, then a separate analysis will also be carried out for multiparous women, and no combined analysis undertaken.

Assessment of Test Characteristics: One of the aims of this study is to assess the test characteristics of SE-CAD for the prediction of PTB in a cohort of pregnant women.

In this assessment, we will first look at SE-CAD as a continuous measure. Smooth plots of the association between PTB outcome and SE-CAD are generated using methods for generalized additive models to help evaluate linearity and choice of cut-off. We will also assess potential cut-offs using ROC methods. To do this, the novel biomarker is classified as either “positive” or “negative” using varying cut-point; a 2×2 table is constructed and the test characteristics defined as follows for each cut-point: We also construct 95% confidence intervals around our estimates of the test characteristics. See simplification paragraph below. We focus on sensitivity as the primary outcome of interest.

Multivariable Predictive Model: Logistic regression will also be used to develop a predictive model for PTB, which will serve as the basis for a clinical predictive index. Each of the three time periods are analyzed as separate models, before looking at longitudinal models (see below). The three models are compared for predictive ability in order to determine if one time frame is the most useful for prediction. For the initial models, we will enter all risk factors that will have shown in the bivariate analysis at least borderline associations with PTB (p<0.20). Terms will then be removed from the model using a backward elimination strategy, removing those factors whose deletion does not cause a significant decrease in the ROC area, see section titled “Comparison of Models Using ROC Analysis” below. For each model under consideration a score for each person is derived using the actual values of the regression coefficients. The goal is to utilize all variables that improve the discrimination of the model. Unlike an explanatory model, whose goal is to determine which potential risk factors are independently associated with PTB, the predictive models are developed in order to determine an optimal combination of variables that best predicts the outcome. Particular attention is given to variables as modifiers of the effect of SE-CAD, or other risk factors. We have determined a priori that four interactions with SE-CAD are of interest in our secondary analyses: SE-CAD and BV, cervical length, race and physical cervical dilation. Logistic regression is use to test for a significant interaction between SE-CAD and each of these variables. Where appropriate, separate predictive models may be generated based on the interaction results to facilitate clinical use; e.g. models by race. While it is important to include all potentially predictive variables and interactions, there is the possibility that variables are included solely on the basis of the multiple comparisons in the study (i.e., by chance). Variables and interactions included in the model must be biologically plausible for PTB. That is, statistical inference must correspond to common sense; conclusions that do not agree with medical knowledge or reasoning are likely to be false and should be viewed with skepticism. Of course, the variables that are entered into the model have been selected because they have been hypothesized as risk factors in prior studies or are possible risk factors for repeat cesarean section. Nonetheless, as an initial attempt to minimize Type I errors in variable selection, the number of variables potentially eligible for entry into the model is restricted such that there are at least 10 cases of PTB for each candidate variable, after screening with bivariate analysis.

Comparison of Models Using ROC Analysis: The decision rules derived from SE-CAD and risk factors developed using logistic regression are evaluated as diagnostic tests. For each “decision rule”, the sensitivity of a predictive function is defined as the proportion of subjects with an event (in this study, PTB) who are also correctly predicted to have the event by the rule. Specificity is defined as the proportion not developing the endpoint that also has a negative test. Multiple test cutoff points can be defined from each model. Each of these cutoffs is associated with a true positive rate (TPR) and false positive rate (FPR) based on actual outcome. Receiver-Operator Characteristic (ROC) curves, in which the TPR and FPR for various cutoff points are plotted, are used to graphically describe the relationship between various cutoffs and the associated test characteristics for the models and biomarkers. These ROC analyses are used in conjunction with assumptions or measurements regarding the relative benefits and burdens of true positives, true negatives, false positives, and false negative. We will evaluate calibration (i.e., the agreement between the predicted probability and the observed probability of cesarean section) using the Homer-Lemeshow statistic and by computing model accuracy. The area under the ROC curve measures the ability of a model to discriminate between those with and without the outcome—PTB. ROCs are used to evaluate and compare the discrimination of the various models and tests. Specifically, given a randomly chosen pair of individuals, one of whom has PTB and one who does not, the ROC area measures the probability that the case is assigned a higher risk score than the control. We plan specifically to compare the ROC curves generated for (1) the full clinical predictive index, (2) the clinical predictive index excluding SE-CAD, (3) the clinical predictive index excluding cervical length and (4) SE-CAD alone. We believe these comparisons are essential, and will give great insight into which variable, or combination of variables, leads to the greatest discrimination. In this case, we need to utilize special methods to account for the correlation between ROC curves. An analytical comparison between models is done using the C-index. This method examines all discordant pairs of outcomes for each subject, and uses a scoring system to weigh outcomes based upon the value of the prognostic model. The resulting statistic is a ratio between the score and the number of discordant outcomes. An estimate of the standard error for this ratio has been suggested by Hanley and McNeil, and can be used to calculate a z-statistic to test the equivalence of two C-indices derived from the same study subjects.

Simplification: To be useful, a clinical rule must be easily applied. In order to ensure easy understanding and unhindered application, the variables and parameters comprising the predictive score is simplified with two methods: 1) by recalculating the predictive score using values of the coefficients, rounded to the nearest integer, and 2) by calculating the score simply as the sum of the dichotomous “risk factors” identified by the model. (It is necessary, for this approach, to recode continuous predictors as dichotomous variables.) This results in the simplest possible model. The performance of the new rules are compared with that of more complete models.

Internal Validation of the Prediction Rule: The prediction rule is internally validated with a “bootstrapping” approach. With this approach, a large number of samples are taken from the original data (with replacement) and the predictive accuracy (percent correct and C-index) is computed for each of these samples. Sampling with replacement essentially generates a population identical in composition to the sample. This method provides a relatively unbiased estimate of the test characteristics of the predictive rule, however, further validation is required with additional prospectively collected data as this is the gold standard for evaluation of promising markers. The ultimate goal of this exercise is to formulate the most valid yet clinically useful model.

Longitudinal Model: Since SE-CAD is collected at three time points (16-20, 20-24, 24-28 weeks) we also utilize methods for longitudinal data. This allows us to use all available data on a subject while accounting for the inherent correlation between measurements on the same woman. Additionally, the combined information may allow us to see patterns over time that are not evident within the individual time points, and may aid in prediction. The methodology of Generalized Estimating Equations (GEE) is used to evaluate changes over time via logistic models for PTB.³⁶ Additionally, when examining the secondary outcome of days to delivery from SE-CAD collection, linear mixed effects models in which random effects due to subject and/or time are included to account for the correlation among repeated observations on each subject are used.³⁷

### Example 8

### Analysis of Novel Immune Biomarkers in Predicting PTB

The use of biomarkers in preterm birth (PTB) has focused on inflammatory pathways. This focus is validated considering the presumed pathogenesis in many cases of spontaneous preterm birth. To date, the focus has been on cytokines which has demonstrated little clinical promise for prediction of PTB. The inventors investigated whether novel biomarkers in immune pathways were associated with PTB in a cohort of high-risk women.

A prospective cohort of women with preterm labor was used (N=110). Women with singleton pregnancies between 22-33 weeks were eligible. Maternal serum was obtained at time of enrollment. Gestational age at delivery and pertinent maternal information was obtained. Soluble forms of early leukocyte activation and cellular adhesion molecules were assessed by a bead-assay (sE-Selectin; sP-selectin; sICAM; sVCAM). Markers of innate immunity (LAL-presence of LPS; sCD14, and pentraxin) were assessed by standard ELISA. Mean levels of each analyte were compared between women with and without PTB using T-test or Mann Whitney Rank Sum. Cut-off points at the median level for each biomaker (low/high) in the whole cohort were used and associations between cut-points with outcomes were analyzed by Chi Square analysis.

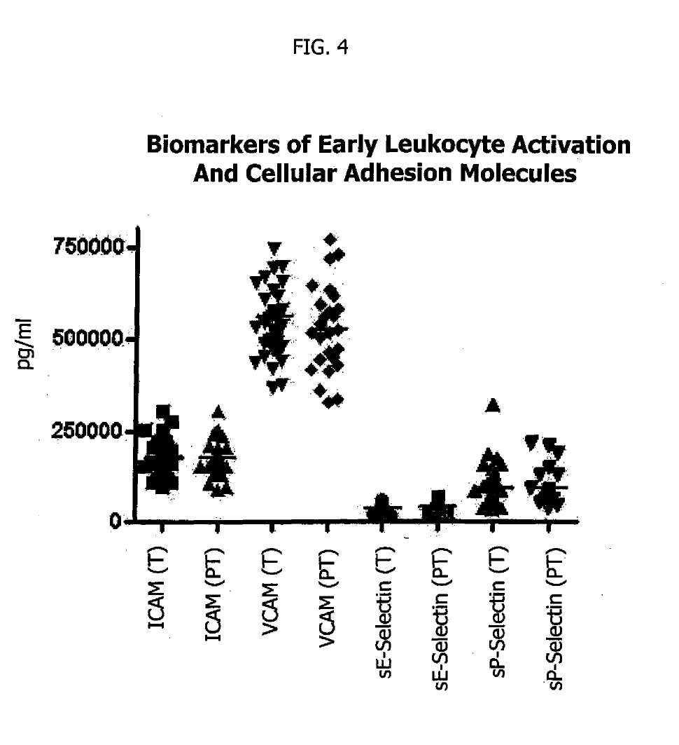

LAL, sCD14, pentraxin, sE-selectin, sP-selectin, slCAM or sVCAM levels were not significantly different in those women who had a preterm or term birth (FIG. 4). None of the biomarkers were associated with clinical or histological chorioamnionitis or with adverse neonatal outcomes.

Similar to assessment of cytokines in maternal serum, these immune biomarkers in innate immune, early leukocyte activation and soluble cellular adhesion molecules do not appear to accurately predict which women will deliver preterm. However, in PTB, activation of immune pathways may be limited to the uterus and thus, systemic markers of these pathways in the mother may prove to have limited clinical applications. These data indicate that, unlike SE-CAD, these inflammation biomarkers do not accurately predict pre-term birth.

Each and every patent, patent application, and publication, including websites cited herein, and the provisional U.S. patent application No. 61/256,066, is hereby incorporated herein by reference in its entirety. While this invention has been disclosed with reference to specific embodiments, it is apparent that other embodiments and variations of this invention are devised by others skilled in the art without departing from the true spirit and scope of the invention. The appended claims include such embodiments and equivalent variations.

### PUBLICATIONS

1. WOOD N S, et al. The EPICure study: associations and antecedents of neurological and developmental disability at 30 months of age following extremely preterm birth. Arch Dis Child Fetal Neonatal Ed 2005;90:F 134-40.

2. RICH-EDWARDS J, et al. Maternal experiences of racism and violence as predictors of preterm birth: rationale and study design. Paediatr Perinat Epidemiol 2001;15 Suppl 2:124-35.

3. DOMINGUEZ T P. Race, racism, and racial disparities in adverse birth outcomes. Clin Obstet Gynecol 2008;51:360-70.

4. GISCOMBE C L, LOBEL M. Explaining disproportionately high rates of adverse birth outcomes among African Americans: the impact of stress, racism, and related factors in pregnancy. Psychol Bull 2005;131:662-83.

5. LI D, et al. Presence of depressive symptoms during early pregnancy and the risk of preterm delivery: a prospective cohort study. Hum Reprod 2009;24:146-53.

6. GOLDENBERG R L, et al. The Preterm Prediction Study: sequential cervical length and fetal fibronectin testing for the prediction of spontaneous preterm birth. National Institute of Child Health and Human Development Maternal-Fetal Medicine Units Network. Am J Obstet Gynecol 2000;182:636-43.

7. IAMS J D, et al. The length of the cervix and the risk of spontaneous premature delivery. National Institute of Child Health and Human Development Maternal Fetal Medicine Unit Network. N Engl J Med 1996;334:567-72.

8. FONSECA E B, et al. Progesterone and the risk of preterm birth among women with a short cervix. N Engl J Med 2007;357:462-9.

9. IAMS J D, et al. The preterm prediction study: can low-risk women destined for spontaneous preterm birth be identified? Am J Obstet Gynecol 2001;184:652-5.

10. MEIS P J, et al. The preterm prediction study: significance of vaginal infections. National Institute of Child Health and Human Development Maternal-Fetal Medicine Units Network. Am J Obstet Gynecol 1995;173:1231-5.

11. TOLOSA J E, et al. The International Infections in Pregnancy (IIP) study: variations in the prevalence of bacterial vaginosis and distribution of morphotypes in vaginal smears among pregnant women. Am J Obstet Gynecol 2006;195:1198-204.

12. NELSON D B, et al. Self-collected versus provider-collected vaginal swabs for the diagnosis of bacterial vaginosis: an assessment of validity and reliability. J Clin Epidemiol 2003;56:862-6.

13. CAREY J C, et al. Metronidazole to prevent preterm delivery in pregnant women with asymptomatic bacterial vaginosis. National Institute of Child Health and Human Development Network of Maternal-Fetal Medicine Units. N Engl J Med 2000;342:534-40.

14. GONZALEZ J M, et al. Preterm and Term Cervical Ripening in CDl Mice (Mus musculus): Similar or Divergent Molecular Mechanisms? Biol Reprod 2009.

15. XU H, et al. Preventing cervical ripening: the primary mechanism by which progestational agents prevent preterm birth? Am J Obstet Gynecol 2008;198:314 el-8.

16. ELOVITZ M, WANG Z. Medroxyprogesterone acetate, but not progesterone, protects against inflammation-induced parturition and intrauterine fetal demise. Am J Obstet Gynecol 2004;190:693-701.

17. ELOVITZ M A, GONZALEZ J. Medroxyprogesterone acetate modulates the immune response in the uterus, cervix and placenta in a mouse model of preterm birth. J Matern Fetal Neonatal Med 2008;21:223-30.

18. ELOVITZ M A, MRINALINI C. Animal models of preterm birth. Trends Endocrinol Metab 2004;15:479-87.

19. GONZALEZ J M, et al. Maternal mortality from systemic illness: unraveling the contribution of the immune response. Am J Obstet Gynecol 2009;200:430 el-8.

20. ELOVITZ M, MRINALINI C. Preterm labor and 17-P: lessons from a mouse model. American Journal of Obstetrics and Gynecology 2006;193:S49.

21. ELOVITZ M A et al. A new model for inflammation-induced preterm birth: the role of platelet-activating factor and Toll-like receptor-4. Am J Pathol 2003;163:2103-11.

22. MUISE A M, et al. Polymorphisms in E-cadherin (CDHl) result in a mislocalised cytoplasmic protein that is associated with Crohn's disease. Gut 2009;58:1121-7.

23. MA X, et al. DNA polymorphisms in exon 1 and promoter of the CDHl gene and relevant risk of transitional cell carcinoma of the urinary bladder. BJU Int 2008;102:633-6.

24. SHIN Y, et al. A functional polymorphism (−347 G-->GA) in the E-cadherin gene is associated with colorectal cancer. Carcinogenesis 2004;25:2173-6.

25. NUGENT R P, et al. Reliability of diagnosing bacterial vaginosis is improved by a standardized method of gram stain interpretation. J Clin Microbial 1991;29:297-301.

26. SHAH S J, et al. Identification and quantification of preterm birth biomarkers in human cervicovaginal fluid by liquid chromatography/tandem mass spectrometry. J Proteome Res 2009;8:2407-17.

27. BASTEK J A, et al. Do Neonatal Outcomes Differ Depending on the Cause of Preterm Birth? A Comparison between Spontaneous Birth and Iatrogenic Delivery for Preeclampsia. Am J Perinatol 2009.

28. BASTEK J A, et al. Adverse neonatal outcomes: examining the risks between preterm, late preterm, and term infants. Am J Obstet Gynecol 2008;199:367 el-8.

29. DEAVE T, et al. The impact of maternal depression in pregnancy on early child development. Bjog 2008;115:1043-51.

30. KORJA R, et al. Maternal depression is associated with mother-infant interaction in preterm infants. Acta Paediatr 2008;97:724-30.

31. SU K P, et al. Different cutoff points for different trimesters? The use of Edinburgh Postnatal Depression Scale and Beck Depression Inventory to screen for depression in pregnant Taiwanese women. Gen Hosp Psychiatry 2007;29:436-41.

32. MATTHEY S, et al. Variability in use of cut-off scores and formats on the Edinburgh Postnatal Depression Scale: implications for clinical and research practice. Arch Womens Ment Health 2006;9:309-15.

33. KRIEGER N, et al. Experiences of discrimination: validity and reliability of a self-report measure for population health research on racism and health. Soc Sci Med 2005;61:1576-96.

34. KRAMER M R, HOGUE C R. What Causes Racial Disparities in Very Preterm Birth? A Biosocial Perspective. Epidemiol Rev 2009.

35. SRINIVAS S K, et al. Periodontal disease and adverse pregnancy outcomes: is there an association? Am J Obstet Gynecol 2009;200:497 el -8.

36. ZEGER S L, LIANG K Y. Longitudinal data analysis for discrete and continuous outcomes. Biometrics 1986;42:121-30.

37. LAIRD N M, WARE J H. Random-effects models for longitudinal data. Biometrics 1982;38:963-74.

What is claimed is:

1. A method for diagnosing, or predicting the likelihood of occurrence of, pre-term birth (PTB) comprising: measuring the level of expression of a biomarker soluble E-cadherin (SE-CAD) in a biological sample from a pregnant mammalian subject, wherein an increased level of expression of SE-CAD above the level of expression in a predetermined control is an indication of a diagnosis of increased risk of pre-term birth.

2. The method of claim 1, wherein said predetermined control is a level of SE-CAD in a biological sample from a member selected from the group consisting of
(a) a healthy pregnant mammalian subject at the same time of pregnancy as the subject;
(b) a healthy pregnant mammalian subject who did not develop pre-term birth;
(c) a population of multiple subjects (a) or (b); and
(d) the same subject at an earlier time in the pregnancy.

3. The method of claim 2, wherein the level is a mean or average, a numerical mean or range of numerical means, a numerical pattern, a graphical pattern or an expression profile.

4. The method according to claim 1 wherein said biological sample is selected from the group consisting of serum, urine, and cervicovaginal fluid.

5. The method according to claim 1, wherein said measuring step comprising measuring the expression of SE-CAD as nucleic acid or protein.

6. The method according to claim 1, further comprises measuring the level of expression of at least one additional biomarker of PTB in said sample, wherein the combined changes in expression of SE-CAD and the additional biomarker from their respective levels of expression in the predetermined control is an indication of a diagnosis of increased risk of PTB.

7. The method according to claim 6, wherein said additional PTB biomarker is fetal fibronectin in maternal serum.

8. The method according to claim 1, wherein the mammalian subject providing the biological sample has clinical symptoms selected from the group consisting of bacterial vaginosis, elevated FFN, short cervical length, stress, depression, inflammation.

9. The method according to claim 1, wherein the subject's biological sample is obtained or after 16 weeks of pregnancy.

10. The method according to claim 1, further comprising repeating said measuring of SE-CAD levels multiple times during the subject's pregnancy.

11. The method according to claim 1, further comprising measuring SE-CAD levels in a series of subject samples taken at different times during the pregnancy and identifying a pattern of increased expression of SE-CAD throughout the pregnancy.

12. The method according to claim 1, wherein said subject is being treated for increased likelihood of PTB and wherein the method enables a determination of the efficacy of the treatment.

13. The method according to claim 1, further comprising contacting a biological sample from a subject with a diagnostic reagent that measures a first level of soluble E-cadherin (SE-CAD) nucleic acid or protein in said sample.

14. The method according to claim 2, further comprising: contacting a second biological sample from the subject at a second later time during the pregnancy and measuring a second level of SE-CAD; and providing a diagnosis of increased risk of pre-term birth based upon said an increase in the second level over a level of SE-CAD in a first biological sample from a member selected from the group consisting of:
(i) a healthy pregnant mammalian subject at the same time of pregnancy as the second sample from the subject;
(ii) a healthy pregnant mammalian subject who did not develop pre-term birth;
(iii) a population of multiple subjects (a) or (b); and
(iv) the same subject at an earlier time in the pregnancy.

15. The method according to claim 14, wherein the subject has an increased risk of developing pre-term birth if the second SE-CAD sample level is higher than any of levels (i) through (iv).

16. The method according to claim 13, wherein said contacting comprises forming a direct or indirect complex in said biological sample between a diagnostic reagent for SE-CAD and the SE-CAD nucleic acid or protein in the sample.

17. The method according to claim 16, wherein said contacting further comprises measuring a level of the complex in a suitable assay.

18. The method according to claim 16, wherein the diagnostic reagent is labeled with a detectable label.

19. The method according to claim 18, wherein said label is an enzyme, a fluorochrome, a luminescent or chemi-luminescent material, or a radioactive material.

20. The method according to claim 16, wherein the diagnostic reagent is an antibody or fragment thereof specific for SE-CAD.

21. The method according to claim 13, wherein the diagnostic reagent is selected from the group consisting of (a) a polynucleotide or genomic probe that hybridizes to SE-CAD cDNA or mRNA; (b) a PCR primer-probe set that amplifies and detects a polynucleotide sequence of SE-CAD mRNA.

22. The method according to claim 13, wherein said reagent is immobilized on a substrate.

23. The method according to claim 13, wherein the diagnostic reagent comprises a microarray, a microfluidics card, a computer-readable chip or chamber.

24. The method according to claim 13, wherein the diagnostic reagent enables detection of changes in expression in SE-CAD in the subject's biological sample from that of a reference expression profile, said changes correlated with the likelihood of PTB.

25. The method according to any of claims 1-24, wherein said measuring is performed by a computer processor or computer-programmed instrument that generates numerical or graphical data useful in diagnosing the likelihood of PTB.

26. The method of claim 1, further comprising coupling the relationship of the sample SE-CAD level with the predetermined control level with the presentation of clinical symptoms, or history of symptoms, of pre-term labor in the subject selected from among FFN, prior PTB, short cervical length, bacterial vaginosis, maternal/uterine infection or inflammation, smoking, sexually transmitted diseases, African American race, low socio-economic status, stress, and depression

27. The method according to claim 1, further comprising coupling the comparative relationship of the sample SE-CAD level with the predetermined control with a history of pre-term birth.

28. The method according to claim 1, which provides a quantitative assessment of the likelihood or risk of pre-term birth in a subject who has not yet developed clinical symptoms of pre-term labor.

29. A method for screening a population of pregnant women for premature cervical remodeling comprising: measuring the level of expression of a biomarker soluble E-cadherin (SE-CAD) in a biological sample from a pregnant mammalian subject, wherein an increased level of expression of SE-CAD above the level of expression in a predetermined control is an indication of premature cervical remodeling.

30. Use of a diagnostic reagent that detects soluble E-cadherin (SE-CAD) nucleic acid or protein in a sample for the diagnosis of PTB.
